(19) United States
(12) Patent Application Publication
(10) Pub. No.: US 2015/0260616 A1
(43) Pub. Date: Sep. 17, 2015

(54) Devices for Automated Sample Collection, Quantificatoin, and Detection for Insect Borne Bio-Agent Surveillance

(71) Applicant: Board of Trustees of the Leland Stanford Junior University, Palo Alto, CA (US)

(72) Inventors: Manu Prakash, Stanford, CA (US); Haripriya Mukundarajan, Stanford, CA (US); Margreth Mpossi, Stanford, CA (US); Nate Cira, Stanford, CA (US)

(21) Appl. No.: 14/423,383

(22) PCT Filed: Aug. 27, 2013
(86) PCT No.: PCT/US13/56843

§ 371 Date: Feb. 23, 2015

Related U.S. Application Data
(60) Provisional application No. 61/693,745, filed on Aug. 27, 2012.

(51) Int. Cl. G01N 1/10; C12Q 1/68; G01N 33/569

(52) U.S. Cl. CPC G01N 1/10; G01N 33/56966; C12Q 1/6806; G01N 2001/1031; G01N 2333/43591; G01N 2333/4353; G01N 2333/185

(57) ABSTRACT

Devices and methods for insect surveillance are disclosed. In particular, the invention relates to devices for collecting samples of salivary fluid from insects and multiplexed high-throughput methods of screening insect samples for specific insect species, genetically modified strains, and insect-borne pathogens. An attractant is used to induce insects to deposit salivary droplets in a 2-dimensional microfluidic array having media-filled pockets sized to collect saliva from a single insect. The array for sample collection can be integrated with a microfluidic device for high-throughput processing of insect samples. The microfluidic device can be designed to perform multiplexed PCR or immunoassays, for example, for insect genotyping or pathogen detection. Devices can be used for screening populations of insects for arthropod-borne diseases, studying genetically modified populations released into the wild, determining the presence and quantity of specific arthropod species and pathogens, and delivery of bioagents.

### RELATED APPLICATIONS

This application claims priority to U.S. Patent Application Ser. No. 61/693,745, filed Aug. 27, 2012, and International Patent Cooperation Treaty Application Serial No. PCT/US13/56843, filed Aug. 27, 2013, which are incorporated herein in its entirety by this reference.

### BACKGROUND OF THE INVENTION

The present invention pertains generally to devices and methods for insect surveillance. In particular, the invention relates to devices for collecting samples of salivary fluid from insects and multiplexed high-throughput methods of screening insect samples for specific insect species, genetically modified insect strains, and insect-borne pathogens.

There are roughly 200 million insects per person on our planet. More than a trillion mosquitoes with nearly 3,500 distinct species are alive at a given moment. A single droplet of saliva deposited during a bite by a mosquito is enough to cause a severe case of malaria. Currently, more than half the world's population is at risk of contacting lethal human diseases from insect bites, including malaria, sleeping sickness, Chikungunya fever, Dengue, West Nile encephalitis, Yellow fever, Lyme disease, Tularaemia and many others. The impact of environmental changes on this complex interaction between vectors, parasites and humans is highly uncertain and has been extremely difficult to measure. This takes a huge toll on the human population, specifically in developing countries, where malaria alone causes 300 million cases of infection with more than a million deaths per year. In a medical context, no effective drugs or vaccines are currently available for diseases like Dengue, and timely availability of effective drugs for malaria is questionable. Thus it becomes critical to tackle disease vectors directly and hence predict ecological factors. Moreover, large-scale ecological perturbations such as: (i) Overuse of insecticides leading to chemical resistance; (ii) emergence of new parasites; (iii) release of transgenic insects, sterile insects or insects infected with pathogen-suppressing endosymbionts like Wolbachia that have the capacity to influence the population structure of wild insects but require field monitoring; (iv) impact of human activity on global climate; and (v) predictions of climate change; each of these factors have a significant but unpredictable influence on vector populations which can only be untangled by high throughput field measurements. For example, currently, no model or equation can capture how rise of global temperatures will affect spread of malaria or why we have an enormous resurgence of diseases such as yellow fever, malaria and onchocerciasis that were previously thought to be under control 30 years ago.

On the more basic science level, due to lack of measurement tools, very little is known about ecological factors that influence vector-parasite interactions [Shahabuddin, M., Costero, A. Spatial distribution of factors that determine sporogonic development of malaria parasites in mosquitoes. Insect Biochem Mol Biol 31 (2001) 241-240; Boëte, C. Malaria parasites in mosquitoes: laboratory models, evolutionary temptation and the real world. Trends Parasitol. (2005) 21: 445-447]. Though lab model systems exist [Vaughan, J. A., Noden, B. H. and Beier, J. C. Population dynamics of Plasmodium falciparum sporogony in laboratory-infected Anopheles gambiae. J. Parasitol. (1992) 78: 716-724], ecological pressures placed on an insect vector population with a human disease reservoir are impossible to replicate in the lab. Big questions in vector ecology that are simple to ask but very hard to answer with existing tools include: Where are the vectors and how many? How many of them are infected? Where do they go? How far do they spread? What happens with seasonal weather changes? Where is malaria in the winter when the vector population has a temporarily collapsed?

No high-throughput tools currently exist to monitor vector populations in field settings. Current surveillance techniques for identifying human biting traits and mosquitoes involves manual capture (using humans as live bait), a method that is 100 years old, extremely inefficient and hard to justify on ethical grounds. Individual mosquitoes are manually dissected to isolate salivary glands that are imaged for presence of parasites such as Plasmodium. Several PCR-based techniques use extracts from hundreds of ground-up mosquitoes that do not exclusively detect the presence of transmissible pathogens specifically in mosquito saliva, and consequently overestimate infection rates. With typical infection rates being only about 1 infected mosquito out of 1000 even in malaria endemic regions, it is almost impossible to get an accurate picture of disease distribution at a high resolution (which requires dissecting 10-50,000 mosquitoes per site).

There remains a need for new methods and devices for screening insects, in particular for detecting arthropod borne diseases, studying genetically modified populations released into the wild, determining the presence and quantity of specific arthropod species, and for delivery of bioagents.

### SUMMARY OF THE INVENTION

The invention relates to devices, systems and methods for insect surveillance. In particular, the invention relates to devices and methods for performing collection, quantification, and detection of insect borne bio-agents. In certain embodiments, the devices can be used to detect arthropod borne diseases, study genetically modified populations released into the wild, determine the presence and quantity of specific arthropod species, or for delivery of bioagents.

The present invention includes a multiplexed high-throughput method of collecting samples of salivary fluid from insects and analyzing them through a variety of microfluidic assays. The basic idea of the invention is a device that collects and stores saliva samples released when an insect bites into it. The device is baited with the appropriate chemicals and temperature to attract the insect of interest.

Current state-of-the-art in this area includes capturing insects (mainly mosquitoes) using human bait, or CDC traps, and subsequently analyzing them by dissection. Dissection is a problem because of the skilled manpower and time required to dissect and microscopically analyze the large number of insects required to derive population statistics (˜50,000 per site per time period). However, in addition to dissection, PCR and ELISA based methods are also used. The drawback in this case is cost—these involve expensive reagents that cannot be used for large scale nationwide surveillance, for instance. Also, if each sample out of about 50,000 has to be processed independently, the pipetting involved requires prohibitively high levels of trained manpower. This device represents more than a 1000-fold increase in sample collection and processing efficiency with a corresponding reduction in cost and manpower requirement, thus forming a cost-effective solution to disease surveillance at national or global scales.

The device can take a variety of physical forms. For example, a shallow dish of oil with surfactant added to stabilize salivary droplets, after which salivary droplets can be post-processed using droplet microfluidic techniques. Another form is a high-density array of pockets sized to isolate exactly one insect bite in each pocket. The reactions for processing are carried out within the pockets themselves, which also behave as nanoliter reaction vessels.

The array can be constructed out of many different materials. For example: (i) it can be machined out of Delrin or a similar plastic; (ii) it can be formed out of polydimethylsiloxane (PDMS) polymer in the manner of conventional microfluidics; (iii) it can be 3-D printed directly; (iv) it can be patterned as hydrophilic and hydrophobic regions on filter paper using methods described in literature; or (v) the pockets can be filled with any medium that facilitates collection and preservation of the droplets, e.g. distilled water, oil, agarose gel, honey, unpolymerized PDMS, waxes and the like.

Processing techniques that can be applied to analyze the samples include a variety of assays. For example: (i) Multiplexed PCR assays, where probes with a variety of fluorophores are used to detect insect vector DNA, pathogen DNA, or DNA of any insect microbiota of interest, wherein multiple specific sub-species of insect vectors or pathogens can be selectively identified, or probe sequences common to all insects/pathogens of that genus/species can be used as a broad screen; (ii) immunoassays can be performed on the samples; and (iii) sequencing techniques can also be applied to sequence DNA present in the captured salivary droplet.

Possible scenarios of use include placing the device in field sites and carrying out processing at the same location using simpler assays (mostly duplexed PCR-based), or placing the device in the field and bringing the collected samples back to the lab for processing using a variety of assays, including multiplexed assays.

An embodiment of the invention focuses on arrays of pockets that are 3-D printed or patterned on paper, with detection of Culex pipiens pipiens mosquitoes and the parasitic filarial nematode Wuchereria bancrofti being the model system for development of duplexed PCR-based assays.

Applications of embodiments of the present invention include: (i) Surveillance of insect borne infectious diseases in the field; (ii) surveillance of the spread and activity of insect disease vectors; (iii) studying the spatial and temporal dynamics of vector-borne infectious disease propagation, to potentially predict epidemics; (iv) monitoring the effectiveness of programs that include the release of bioagents like genetically modified or sterile vectors; (v) large scale collection of insect saliva for vaccine development; (vi) studying the propagation of insecticide resistance in vectors or drug resistance in pathogens; and (vii) identifying the effects of environmental changes (climate, chemical spraying, and the like) on vectors, pathogens and disease propagation.

The vectors and associated diseases that can be studied using this device are most diseases transmitted by haematophagous insects, including, but not limited to: (i) Mosquitoes, which can carry malaria, dengue, West Nile Encephalitis, filariasis, yellow fever, indeed all mosquito-borne arboviral encephalitic diseases; (ii) flies, which can carry sleeping sickness, leishmaniasis, and the like; (iii) fleas, which can carry plague and other diseases; (iv) ticks, which can carry Lyme disease and other diseases; and (v) mites, which can carry scrub typhus and other diseases.

This is the first device that enables high-throughput collection and processing suitable for large scale ecological experiments, while simultaneously achieving a high resolution at the level of single insect bites, which has never been possible before.

These and other embodiments of the subject invention will readily occur to those of skill in the art in view of the disclosure herein.

### BRIEF DESCRIPTION OF THE FIGURES

FIG. 1 is a schematic representation of an insect saliva sample collecting device of the present invention wherein an oil bath is created atop a glass slide inside a silicone gasket and covered with a membrane.

FIG. 2 is a schematic representation of an insect saliva sample collecting device of the present invention wherein an array of wells sized to collect a single insect bite are formed in a substrate and filled with a collection material.

FIG. 3 is a photograph and a schematic representation of an insect saliva sample collecting device of the present invention wherein an array of collection pockets sized to collect a single insect bite are formed in a paper substrate; the device may be printed onto a postcard and used in a citizen science initiative.

FIG. 4 is a schematic representation of a method of detecting genetic material in saliva droplets collected in the insect saliva sample collecting devices of the present invention.

FIG. 5 is a photograph of an electrophoresis gel showing the detection of mosquito (Culex quinquefasciatus) saliva collected in various media through PCR amplification of the acetylcholinesterase 2 (AceII) gene specific to this species. The lanes of the gel correspond to the following samples: (1) ladder, (2) saliva from agarose gel, (3) saliva collected in mineral oil, (4) saliva collected in distilled water, (8) a negative control—distilled water, and (6) a positive control—Culex quinquefasciatus genomic DNA template.

FIG. 6 shows (left to right, top to bottom) (i) collection of salivary samples by forced insect salivation into a capillary containing mineral oil, a close-up view of a saliva trail oozing from a mosquito proboscis inserted in an oil-filled capillary, bites with salivary droplets preserved in 2% agarose gel offered to mosquitoes for 1 hr, images of the gel surface before and after the deposit of saliva and a histogram showing distribution by size of salivary droplets preserved in 2% agarose gel.

FIG. 7 is a schematic representation of a typical scenario of use of the devices and methods of the present invention in the field.

### DETAILED DESCRIPTION OF PREFERRED EMBODIMENTS

The practice of the present invention will employ, unless otherwise indicated, conventional methods of pharmacology, chemistry, biochemistry, microfluidics and recombinant DNA techniques, within the skill of the art. Such techniques are explained fully in the literature. See, e.g., Handbook of Experimental Immunology, Vols. I-IV (D. M. Weir and C. C. Blackwell eds., Blackwell Scientific Publications); A. L. Lehninger, Biochemistry (Worth Publishers, Inc., current addition); Sambrook, et al., Molecular Cloning: A Laboratory Manual (3rd Edition, 2001); Methods In Enzymology (S. Colowick and N. Kaplan eds., Academic Press, Inc.).

All publications, patents and patent applications cited herein, whether supra or infra, are hereby incorporated by reference in their entireties.

### DEFINITIONS

In describing the present invention, the following terms will be employed, and are intended to be defined as indicated below.

It must be noted that, as used in this specification and the appended claims, the singular forms “a”, “an” and “the” include plural referents unless the content clearly dictates otherwise. Thus, for example, reference to “a nucleotide” includes a mixture of two or more nucleotides, and the like.

The term “about,” particularly in reference to a given quantity, is meant to encompass deviations of plus or minus five percent.

Before describing the present invention in detail, it is to be understood that this invention is not limited to particular formulations or process parameters as such may, of course, vary. It is also to be understood that the terminology used herein is for the purpose of describing particular embodiments of the invention only, and is not intended to be limiting.

Although a number of methods and materials similar or equivalent to those described herein can be used in the practice of the present invention, the preferred materials and methods are described herein.

Molecular techniques and genetic engineering have given us many tools to fight vector-borne diseases, using new drugs and genetically engineered sterile vectors. However, we lack the ability to accurately measure their effect on vector and parasite populations on ecological scales. This prevents us from eradicating these diseases in a targeted way. It is therefore critical to understand the dynamics of the relationship between vectors, parasites and humans in field settings, to effectively combat these diseases with modern technology and medicine.

Current surveillance methods cannot be scaled to developing countries with non-existent infrastructure. The present invention provides a scalable method to screen vectors in field settings with costs that are four-orders of magnitude smaller in comparison to conventional methods. The wide applicability of the proposed techniques will allow for quantitative measurement of disease transmission potential amongst vector species, and uncover hidden dynamics in the timing, distribution and abundance of disease competent vectors. If implemented on a global scale, such a surveillance mechanism will significantly increase the capability to combat vector borne diseases and predict epidemics. Rich data sets generated in field settings will thrust infectious disease vector ecology into the genomic age, with the application of molecular techniques on real world samples of vectors and parasites.

Certain mosquito-borne diseases like malaria, dengue and West Nile encephalitis are currently under surveillance in small parts of the world. Typical entomological inoculation rates (EIR=Number of infectious bites/year/person) can be as low as 0.001 even in areas having endemic malaria [J. C. Beier, Methods in Molecular Medicine 72 (2002)]. This requires a huge number of mosquitoes to be tested for an adequate sample of the wild population, particularly for fast mutating parasites like Plasmodium. The current surveillance techniques include manual capture of mosquitoes (which often uses humans as live bait and is ethically questionable) [W. F. Bynum, Science 295, 47-48 (2002)], time-intensive dissection of individual insects (100 per day for an experienced technician) [E. P. Hodgkin, Transactions of the Royal Society for Tropical Medicine and Hygiene 43, 617-634 (1950)] or relatively expensive PCR or ELISA based assays, which are performed at low resolution for pools of up to 50 insects [R. A. Wirtz et al., The American journal of tropical medicine and hygiene 34, 1048-54 (1985)]. Current estimates of saliva production and pathogen transmission rates have been made under conditions of forced salivation in immobilized insects [R. Rosenberg, R. A. Wirtz, I. Schneider, R. Burge, Transactions of the Royal Society for Tropical Medicine and Hygiene 84, 209-212 (1990)] or aggregated in a droplet while feeding [S. L. Anderson, S. L. Richards, C. T. Smartt, Journal of the American Mosquito Control Association 26, 108-111 (2010)., 108-111 (2010)], which cannot be correlated to actual EIR with high confidence. Other vectors like ticks and flies are even less studied in terms of distribution and disease evolution.

The invention relates to devices and methods for insect surveillance. Arthropods transmit a variety of diseases and are destructive to many crops. Current methods for identifying and quantifying insects are tedious, time consuming, require high levels of expertise, and are often only applicable in a narrow range of contexts. The inventors describe novel devices for high throughput arthropod screening. An attractant is used to induce insects to deposit salivary droplets in an insect sampling area such as wells, for example hydrogel pockets, sized to collect saliva from a single insect. The devices, which comprise a plurality of pockets or wells organized in a 2-dimensional array, are designed to collect numerous samples at discrete locations for subsequent analysis in parallel. In particular, the array for sample collection can be integrated with a microfluidic device for high-throughput processing of insect samples. The microfluidic device can be designed to perform multiplexed PCR or immunoassays, for example, for insect genotyping or pathogen detection. Devices can be used for screening populations of insects for arthropod-borne diseases, studying genetically modified populations released into the wild, determining the presence and quantity of specific arthropod species, and delivery of bioagents. Additionally, data generated by devices will allow early detection of insect-borne pathogens, establishment of thresholds for action, for example, to prevent disease transmission or crop damage, and evaluation of the efficacy of current insect control methods.

In order to further an understanding of the invention, a more detailed discussion is provided below regarding devices and methods for insect surveillance.

### EXPERIMENTAL

Below are examples of specific embodiments for carrying out the present invention. The examples are offered for illustrative purposes only, and are not intended to limit the scope of the present invention in any way.

Efforts have been made to ensure accuracy with respect to numbers used (e.g., amounts, temperatures, etc.), but some experimental error and deviation should, of course, be allowed for.

### Example 1

### Oil-Filled Cavity Insect Saliva Sample Collecting Device

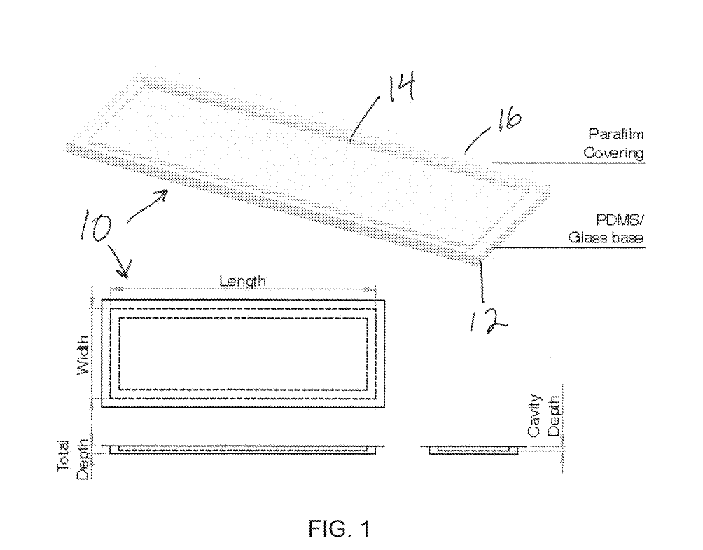

In perhaps the simplest embodiment, the present invention comprises an oil-filled cavity device 10 (FIG. 1) for collecting insect saliva samples. A substrate 12, which in a preferred embodiment, is a glass slide, supports the oil-filled cavity formed by an enclosed gasket 14 which creates a shallow enclosure that is filled with a fluid, such as mineral oil. The oil can be substituted with any medium that facilitates collection and preservation of the droplets, for example, distilled water, glycerol, agarose gel, honey, unpolymerized polydimethylsiloxane (PDMS) or waxes. The oil is retained inside the enclosure atop the substrate 12 by a covering 16, such as Parafilm® (Bemis Company, WI) which biting insects are able to pierce with their mouthparts to deposit the saliva sample. The gasket 14 can be formed of any suitable material, for example a silicone such as PDMS. The oil-filled cavity insect saliva sample collecting device 10 is mounted to a heater having a temperature sensor and temperature controller (not shown) for maintaining the device 10 at a temperature to attract the insects of interest, commonly human blood temperature of 37° C. Attractants for the target insects may preferably be used with the device 10, including food, odorants, pheromones, and visual lures. Odorants that attract mosquitoes include carbon dioxide, lactic acid and octenol. Insects attracted to the device 10 bite through the covering 16 to deposit saliva in the oil bath where it is preserved as droplets.

In a variation of the above-described device where the oil is replaced with a 1% agarose gel, salivary samples were collected by allowing mosquitoes to feed on a 25 mm×25 mm square 50 of the agarose gel for 2 hours. Over 700 bites were measured and a distribution of bite radius was plotted, with a mean radius of 59 μm. The natural salivary deposits were quantitatively measured to determine the distribution of injected saliva volumes.

### Example 2-2-D

### Array Type Insect Saliva Sample Collecting Device

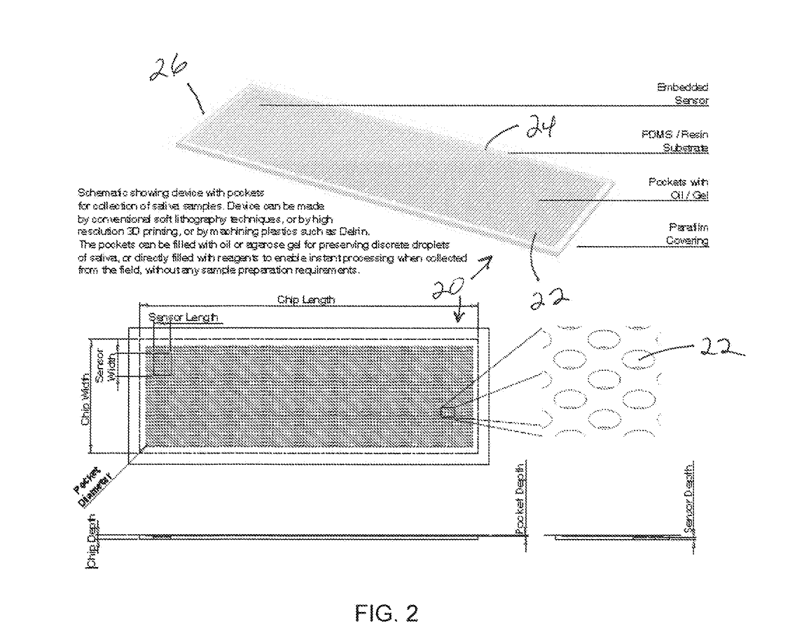

In a preferred embodiment of the present invention, an insect saliva sample collecting device is prepared for collecting samples of saliva deposited by a target insect that has been attracted to the device. One such device is a chip 20 (FIG. 2) which includes an array of wells or pockets 22 formed in substrate 24 and filled with a fluid which collects and retains the saliva deposited by a target insect. The substrate 24 can be made by conventional soft lithography techniques, by high-resolution 3-D printing techniques, or by machining plastics such as Delrin. The fluid filling the pockets or wells 22 is selected to collect, retain and preserve the saliva injected into it by the insect bite. Suitable fluids include oils or gels, such as agarose gel. Alternatively, the wells 22 may be directly filled with reagents to enable instant processing of the saliva samples when collected from the field, thus eliminating a step from the sample preparation process. Preferably, a membrane 26, such as Parafilm®, covers the wells 22 to prevent contamination of the samples and evaporation or other changes in the oil or gel in the wells 22 during the useful life of the chip 20.

### Example 3

### Paper Insect Saliva Sample Collecting Device

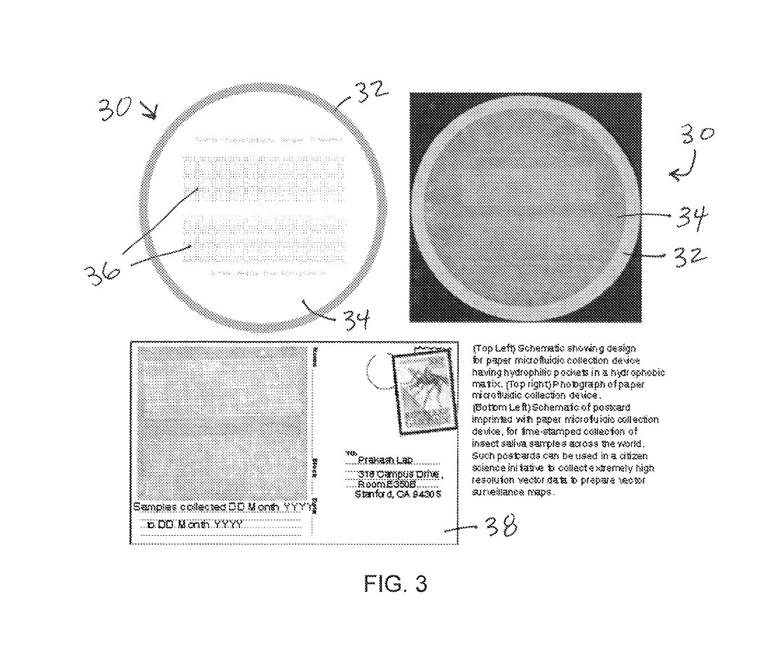

In another preferred embodiment of the present invention, a paper insect saliva sample collecting device 30 (FIG. 3) is created on a paper substrate 32. Paper is a suitable medium for preserving DNA. The paper substrate 32 is coated with a hydrophobic matrix 34 in which is formed an array of hydrophilic pockets 36. The device is made using techniques described in literature for patterning of paper and selectively altering its properties in desired areas to create platforms for low cost assays. In a preferred embodiment, the filter paper 32 is coated with a light-sensitive polymer, exposed to UV through a mask which covers the pockets 36, and then washed in a developing chemical. Accordingly, the unpolymerized polymer in the pockets, which was covered by the mask and not exposed to UV, is washed off. This leaves behind pockets 36 of the original paper 32, embedded in a matrix where the paper 32 is impregnated with the hardened UV-exposed polymer. Process reference [see, A. W. Martinez, S. T. Phillips, G. M. Whitesides, Proceedings of the National Academy of Sciences of the United States of America 105, 19606-11 (2008)]. As a result, the paper collection device is extremely inexpensive. Preferably, a membrane (not shown), covers the pockets 36 to prevent contamination of the saliva collected in the pockets 36 during the useful life of the device 30. An application of the paper device 30 is its incorporation onto a postcard 38 for time-stamped collection of insect samples throughout the world. Postcards 38 can be used in a citizen science initiative to collect extremely high resolution vector data, for example, to prepare vector surveillance maps.

### Example 4

### Method of Detection of Saliva and Genetic Material

Confirmation of the presence of saliva can be done by any method known in the art, such as enzyme-catalyzed precipitation of salts, microscopy or ELISA.

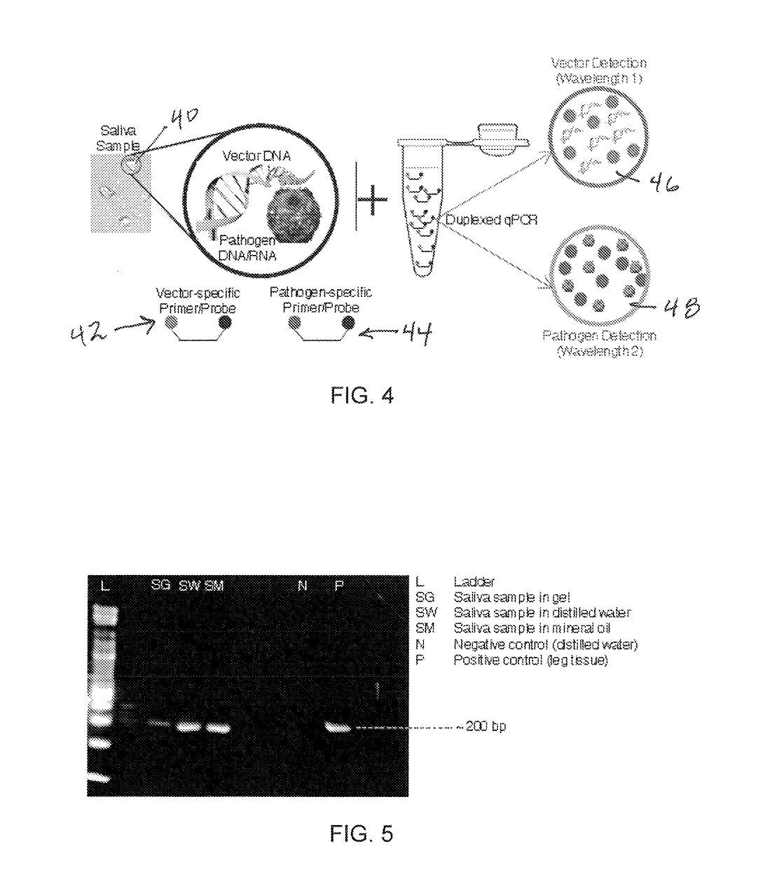

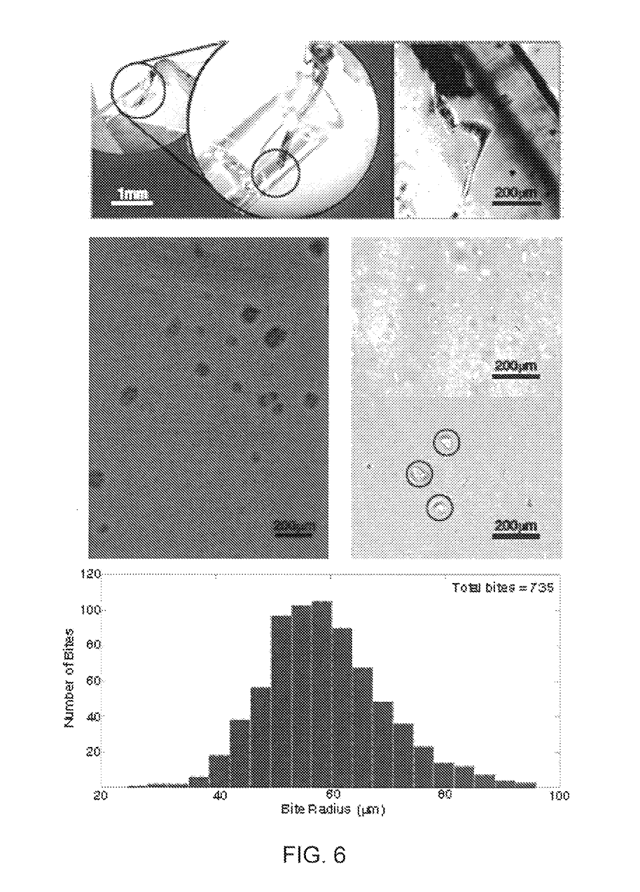

Species identification is achieved by extraction-free PCR that does not require prior DNA purification from the sample. FIG. 4 is a schematic representation of a species identification method using duplexed qPCR. A saliva sample 40 contains DNA from the insect vector and also DNA/RNA from pathogens if the sample comes from an infected vector. Primers specific to the vector 42 and the pathogen 44 are used to amplify specific identifying genetic sequences. In a preferred embodiment, fluorescent probes at different wavelengths are used for each species. Samples from a given species fluoresce at the corresponding wavelength, for example, vector detection 46 is observed at wavelength and pathogen detection 48 is observed at wavelength B. By carrying out this reaction for an array of samples and counting the number of samples fluorescent at each wavelength, the ratio of infected to uninfected samples can be obtained directly. This ratio can directly be correlated to the actual entomological inoculation rate (EIR) and reproduction rate of the parasite population [G. Macdonald, Bulletin of the World Health Organization 15, 613-26 (1956)].

### Example 5

### Microfluidic Device for High-Throughput Insect Vector Analysis

Here we describe the application of high-throughput microfluidic techniques to ecological studies of insect vector-parasite populations. A key idea is to automate large-scale saliva collection and processing in a way that does not perturb insect behavior. The present invention includes a microfluidics device that carries out biochemical assays in low reaction volumes with high quantitative accuracy and that is capable of performing molecular diagnostic analyses of a large collection of saliva samples from different individual insects in parallel. Such a device can be mass-produced for widespread use, saving thousands of hours of manpower and reducing surveillance costs.

A. Hydrogel Array for Collection of Insect Saliva

Collection of Isolated Salivary Droplets and Measurement of Bite Volume

High-throughput collection of salivary droplets was effectively achieved with a hydrogel. Blood-feeding mosquitoes were induced to bite a 1% agarose hydrogel at 37° C. using lactic acid or octenol as bait. Saliva droplets deposited in the agarose gel are detected using a microscope.

Isolation of Individual Bites in a 2-Dimensional High Density Array

Next, we studied insect bites in a 2-D hydrogel array comprising hydrogel pockets of about 150 μm in diameter. The array was fabricated by cutting a pattern on card paper using a laser, treating the paper with SU-8 photoresist to make it hydrophobic, and filling the pockets with 1% agarose gel. The array was made with accurately sized pockets designed to have a high probability of collecting and isolating a single insect bite per pocket. In laboratory tests, we have observed single bite marks in hydrogel pockets. Arrays can be fabricated with over 3000 hydrogel pockets and are capable of processing nanoliter sample volumes on the area of a single microscope slide.

To attract mosquitoes, the array can be baited with thermal and odorant cues and placed in an area of interest for collection of insect samples. Such an array can be used to capture salivary droplets from individual bites from thousands of mosquitoes. In addition, the array can be interfaced with a complementary silicone-based array of reaction wells containing reagents for performing, for example, PCR or immunoassays for analysis of insect samples to identify insect species and detect insects infected with pathogens. It is further possible to apply molecular and sequencing techniques on bite samples to study migratory trends in vectors, evolution of highly mutable parasites, identification of factors influencing insecticide or drug resistance and discovery of new pathogens or biological agents that prevent disease transmission, like Wolbachia [T. Walker et al., Nature 476, 450-3 (2011); R. L. Glaser, M. a Meola, PloS one 5, e11977 (2010)]. Sample processing is possible at a central location with just a thermoelectric plate and microscope, thus, eliminating the need for extensive laboratory facilities.

B. Multiplexed PCR Analysis of Insect Samples

Saliva from each individual bite captured in a hydrogel can be detected, and insects that are infected with pathogens can be identified by duplexed PCR. Taq-Man probes having distinct fluorescence spectra can be used to separately detect insect DNA from each bite and pathogenic genetic material from the bites of infected insects. The two fluorescent readouts from the insect-specific and probes specific to the pathogenic genetic material yield the ratio of bites from infected insects to total number of bites.

In addition, the species of the insect can be determined by amplification of any genetic sequence unique to the insect vector of interet. For example, if the insect vector of interest is Culex quinquefasciatus, species-specific regions of the acetylcholinesterase-2 (Ace2) gene in salivary samples is used [J. L. Smith, D. M. Fonseca, The American journal of tropical medicine and hygiene 70, 339-45 (2004)]. An extraction-free protocol was developed using a Phire Direct PCR Kit (Thermo Fisher Scientific, Vanta, Finland) for direct one step PCR on samples from leg tissue and saliva, without prior DNA extraction steps. Alternately, extraction free PCR was also demonstrated by placing the sample in distilled water prior to adding PCR reagents, or carrying out the reaction with the addition of 1% Triton-X detergent.

C. Microfluidic Device Integrated with Hydrogel Array for Insect Sample

Collection and PCR-Based Analysis

The present invention includes a microfluidic device that integrates sample collection using a hydrogel array with duplexed PCR-based detection. A 2-dimensional array is mated with a microfluidic chip pre-filled with PCR reagents, including primers and fluorescent probes for amplification and detection of insect and pathogen species of interest. The device enables high-throughput multiplexed analysis of insect saliva samples in a single reaction vessel with no sample preparation.

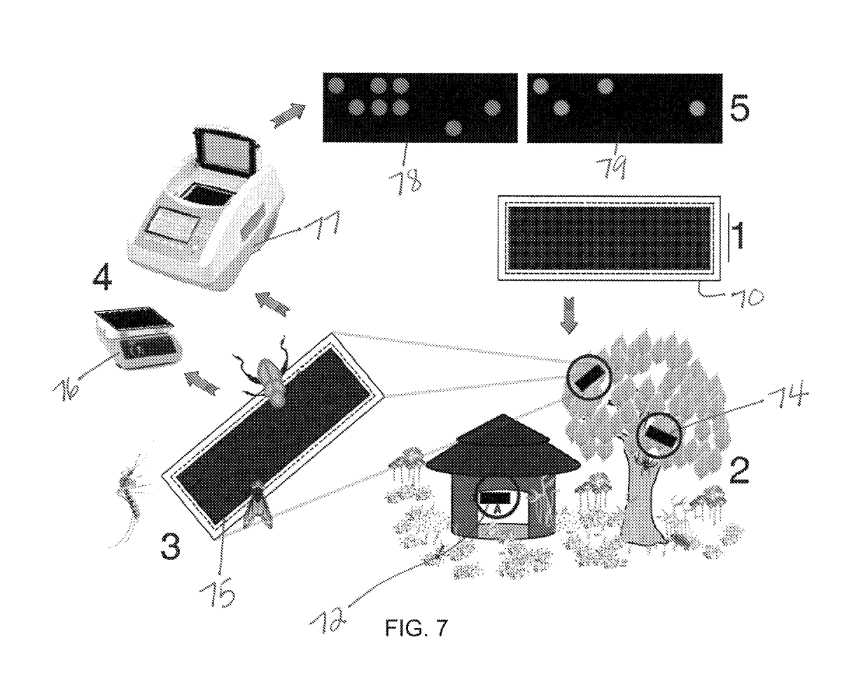

A typical scenario for use of the device is illustrated schematically in FIG. 7. The collection array 70 is prepared loaded with reagents. The array 70 is placed in the field site, both in houses 72 and outside 74 to collect saliva samples from insect vectors of interest. The attracted insects biting the array 75 deposit saliva samples in the wells of the array 70. The array 70 is processed either on-site using a programmed hot plate 76 to carry out duplexed PCR or in a remote facility using more sophisticated techniques 77. The fluorescent readouts from the duplexed PCR show vector bites 78 and infected bites 79.

The prototype device is being tested on Culex pipiens colonies bred in captivity, which have known numbers of infected and uninfected mosquitoes. Culex colonies can be obtained from the laboratory of Manu Prakash and the Centers for Disease Control and Prevention (CDC). In current tests, duplexed PCR is carried out with fluorescent Taq-Man PCR probes for the detection of DNA from Culex pipiens mosquito vectors and Wucherichia bancrofti filarial worms. Statistical correlations between the observed fluorescent readout and mosquito biting patterns, which differ for infected and healthy mosquitoes [P. A. Rossignol, J. M. C. Ribeiro, A. Spielman, The American journal of tropical medicine and hygiene 33, 17-20 (1984)], will be extended to field settings.

The paper device 30 can be simply soaked in the reagent mixture. Alternately, the reagents can be filled into another sample collection device having pockets, and the two devices, one with the field-collected samples and one with the reagents, can be matched to line up their pockets. Alternatively, the mating step can be avoided altogether wherein the chip can having the reagent mixture itself, or a partial set of those reagents which can withstand field conditions, is used as a collection medium instead of gels/oils, since these can also preserve DNA and reduce processing steps.

D. Quantification of infectious West Nile Virus Particles

The prototype microfluidic device can be used to quantify the distribution of infectious WNV particles in a given wild population of Cx. pipiens at a given time. This device can also be used in a novel method to quantify viral pathogens transmitted in single insect bites, which has never been achieved before. The model virus for this is the arbovirus causing West Nile Encephalitis. The device is used to collect samples of mosquito saliva from WNV infected mosquitoes. qRT-PCR using Taq-Man probes can be used to quantify the intensity of fluorescence obtained after amplifying viral genetic material [P.-Y. Shi et al., Journal of Clinical Microbiology 39, 1264-1271 (2001)]. By constructing standard curves to determine the correlation between fluorescence intensity and starting concentration of WNV RNA, the device can be used to quantify viral transmission. Behavioral variations can also be leveraged to preferentially collect samples from species that bite humans and exclude species feeding on other animals or birds. Field testing by screening local mosquitoes for West Nile virus (WNV) will be carried out at Jasper Ridge Ecological Reserve (Stanford), and data is compared with results from the mandatory surveillance of West Nile encephalitis by local vector control districts.

Wild insect population data is collected over several months for Culex vectors and WNV. The study is performed both with high spatial resolution in a region and at high time resolution with tests a week apart. Much previous work has been done on mathematical models of mosquito populations [G. Macdonald, Bulletin of the World Health Organization 15, 613-26 (1956); G. Hasibeder, C. Dye, Theoretical population biology 33, 31-53 (1988); D. L. Smith, F. E. McKenzie, Malaria journal 3, 13 (2004); D. de Souza, T. Tomé, S. Pinho, F. Barreto, M. de Oliveira, Physical Review E 87, 012709 (2013)] using data from captured mosquitoes. In contrast, our study will elucidate trends from data with high spatial and temporal resolution to create a more realistic dynamic model of population interactions, with a particular focus on short-duration perturbations, such as caused by insecticide spraying.

Although current efforts have focused on mosquitoes as the disease vector and, in particular, filarial worms and West Nile virus, future goals include developing assays to screen for other diseases like malaria and dengue, and extending the use of the microfluidic device to screen for other insect species, such as ticks and fleas.

E. Global Insect Surveillance for the Future

The ultimate goal is insect surveillance on a nation-wide scale, which could transform world maps of disease distribution, enabling real-time vector-parasite mapping with resolutions under 1 km² for species distribution studies. Such a large scale insect sampling strategy has never been demonstrated before. Therefore, a method that could identify vector species and the parasites they harbor in a scalable, efficient and cost-effective manner in field settings would be a major breakthrough, providing the ability to study spatial and temporal dynamics of insect-parasite interactions on an ecological scale. In the future, it will be possible to perform worldwide monitoring of diseases such as West Nile encephalitis, malaria, dengue, yellow fever and filariasis, directly in insect populations in field settings. Providing early warning against epidemics and facilitating the development of control strategies targeting specific disease stages can greatly reduce human deaths through preventive action and planned medical care.

What is claimed is:

1. A device for collection and analysis of insect saliva samples comprising:
(a) a support;
(b) a sample collection area attached to the support;
(c) an attractant capable of attracting an insect of interest to the sample collection area wherein the sample collection area retains an insect saliva sample deposited by an insect in the sample collection area; and
(d) a detector for detecting one or more selected attributes of the insect saliva sample.

2. The device of claim 1, wherein said sample collection area is selected from the group consisting of an oil dish, an array of oil-filled wells, a gel dish, an array of gel-filled wells, an array of reagent-filled wells, an array of patterned paper, or other microfluidic sample collection device.

3. The device of claim 2, wherein the wells are sized to correspond to the bite radius of the insect to allow and isolate one insect bite per well.

4. The device of claim 2, wherein the gel is selected from the group consisting of suitable hydrogels, for example agarose and pectin.

5. The device of claim 1, wherein the insect is selected from the group consisting of mosquitoes, fleas, ticks, flies, mites and other insect vectors for diseases passed by bites.

6. The device of claim 1, wherein the attractant is selected from the group consisting of food, odorant, pheromone and visual lure.

7. The device of claim 6, wherein the temperature is maintained by an integrated control system consisting of temperature sensor, heater and microprocessor and powered by batteries for placement in the field.

8. The device of claim 1, wherein the attractant is selected from the group consisting of octenol, R-octenol, Lurex, lactic acid, L-lactic acid, carbon dioxide, methyl eugenol, 1,4-diaminobutane, anisylacetone, p-acetoxyphenylbutanone-2, and t-butyl-2-methyl-4-chlorocyclohexanecarboxylate.

9. The device of claim 1, wherein the attractant is male-specific or female-specific.

10. The device of claim 1, wherein the attractant is species-selective.

11. The device of claim 1, further comprising an insecticide, an insect sterilization agent, or a mating disruption agent.

12. The device of claim 1, further comprising a microfluidic chip comprising:
(a) an array of reaction wells configured such that each reaction well can be connected with an insect sample collection area at the corresponding position in the array, such that when the reaction wells are filled with fluid comprising one or more reagents, the insect sample collection areas are in contact with the one or more reagents; and
(b) a mating microfluidic chip for piping, stamping, soaking or otherwise delivering by contact reagents into sample collection areas prior to processing.

13. The device of claim 12, wherein the reaction wells are filled with reagents for performing PCR, immunoassays, or DNA sequencing.

14. A method for collection and analysis of insect samples using the device of claim 1, the method comprising:
(a) placing the device outside in an area where screening insects is desired;
(b) attracting insects of interest with the attractant;
(c) collecting saliva samples from the insects of interest, wherein each individual insect deposits sliva samples in the insect sampling area; and
(d) analyzing the saliva samples.

15. The method of claim 14, wherein said analyzing step is selected from the group consisting of use of a saliva detection assay, identifying the species of at least one insect that deposited a saliva sample, detecting at least one insect-borne pathogen in the saliva from at least one insect that deposited a saliva sample, detecting a genetic modification in at least one insect that deposited a saliva sample, performing PCR amplification of at least one gene from an insect or insect-borne pathogen, performing an immunoassay to detect at least one antigen from an insect or insect-borne pathogen, and performing DNA sequencing.

16. The method of claim 15, wherein at least one gene comprises a nucleotide sequence unique to and characteristic of all regional variants and natural mutants of the insect of interest.

17. The method of claim 16, wherein the insect is selected from the group consisting of mosquitoes, flies, fleas, ticks, mites and other insect vectors for diseases passed by bites.

18. The method of claim 17, wherein the insect-borne pathogen is any human or animal disease spread by hematophagous insect vectors through saliva.

19. The method of claim 18 wherein the pathogen is selected from the group consisting of malaria, dengue, West Nile encephalitis, yellow fever, filiariasis, elephantiasis, Eastern Equine encephalitis, Lacrosse encephalitis, Saint Louis encephalitis, Japanese encephalitis, heartworm, Rift Valley fever, Chikungunya fever, leishmaniasis, sleeping sickness, scrub typhus, babesiosis, Lyme disease and plague.

20. The method of claim 15, further comprising performing multiple cycles of the method for collection and analysis of insect samples.
